(19) United States
(12) Patent Application Publication
(10) Pub. No.: US 2026/0191946 A1
(43) Pub. Date: Jul. 9, 2026

(54) PORCINE INTERFERON-LIKE RECOMBINANT PROTEIN AND USE THEREOF

(71) Applicant: BODN BIOTECH INC., Richmond (CA)

(72) Inventors: Chunsheng Mao, British Columbia (CA); Shaoyu Li, Henan (CN); Jianyu Fang, Henan (CN); Bin Xu, Henan (CN); Jizhi Li, British Columbia (CA); Jingchuang Chen, Henan (CN); Yale Mao, Henan (CN)

(73) Assignee: BODN BIOTECH INC., Richmond (CA)

(21) Appl. No.: 19/199,397

(22) Filed: May 6, 2025

(30) Foreign Application Priority Data
Jan. 3, 2025 (CN) 202510010370.1

(51) Int. Cl. A61K 39/12; A61K 9/00; A61K 38/00; A61K 39/00; A61K 39/002; A61K 39/09; A61K 39/108; A61K 39/112; A61P 31/14; C07K 14/555; C12N 7/00; C12N 15/85

(52) U.S. Cl. CPC A61K 39/12; A61K 9/0019; A61K 9/0043; A61K 39/002; A61K 39/0258; A61K 39/0275; A61K 39/092; A61P 31/14; C07K 14/555; C12N 7/00; C12N 15/85; A61K 38/00; A61K 2039/53; A61K 2039/542; A61K 2039/543; A61K 2039/552; C07K 2319/20; C12N 2770/10034

(57) ABSTRACT

This invention relates to the field of biomedicine and specifically to a porcine interferon-like recombinant protein and its applications. By constructing and screening a PoIFN-α DNA shuffling bacterial library, a highly active recombinant protein Porferon was obtained. Porferon exhibits potent anti-infective activity against a broad range of porcine pathogens, including viruses, bacteria, and parasites. Its biological activity is significantly superior to that of natural porcine interferon. As such, Porferon can be used in the development of therapeutics for the prevention or treatment of infectious diseases in pigs, offering a novel and effective solution for clinical applications in veterinary medicine.

### CROSS-REFERENCE TO RELATED APPLICATION

This application claims the priority benefit of China application no. 202510010370.1, filed on Jan. 3, 2025. The entirety of each of the above-mentioned patent applications is hereby incorporated by reference herein and made a part of this specification.

### REFERENCE TO A SEQUENCE LISTING

The instant application contains a Sequencing Listing which has been submitted electronically in XML format and is hereby incorporated by reference in its entirety. Said XML copy, created on Apr. 21, 2025, is named 154582_SEQUENCELISTING and is 26,067 bytes in size.

### TECHNICAL FIELD

This invention relates to the field of biopharmaceutical technology, specifically relating to a porcine interferon-like recombinant protein and its applications.

### BACKGROUND OF RELATED ART

Interferons (IFNs) were first discovered in 1957 by Isaacs and Lindenmann as secreted glycoproteins produced by animal cells with antiviral, immunomodulatory, and antitumor effects. They are now recognized as an essential component of the innate immune response (Jonasch E and Haluska F G. Interferon in oncological practice: review of interferon biology, clinical applications, and toxicities. Oncologist. 2001; 6(1):34-55).

Interferons belong to a multigene family and are classified into three subtypes based on gene sequence homology, protein structure, evolutionary relationships, cell surface receptors, and functional characteristics. Type I interferons mainly include IFN-α, IFN-0, IFN—F, IFN-κ, and IFN-ω, which are common across species. Additionally, species-specific interferons include IFN-δ (pigs and horses), IFN-ζ (mice), IFN-τ (cattle), and IFN-ω (pigs, horses, and cattle). Type II interferons consist primarily of a single subtype, IFN-γ. Type III interferons include IFN-λ1 (IL-29), IFN-λ2 (IL-28A), IFN-λ3 (IL-28B), and IFN-λ4.

In pigs, Type I interferons comprise at least 39 functional genes located on chromosomes 1 and 10, including 17 IFN-α subtypes, 11 IFN-δ subtypes, 7 IFN-ω subtypes, and single genes encoding IFN-β, IFN-ε, and IFN-κ.

Initially identified for their antiviral activity, porcine Type I interferons were later found to exhibit antitumor and immunomodulatory effects. Among these, IFN-α and IFN-β subtypes have the most potent antiviral activity. Currently, Type I interferons are widely used in the clinical treatment of human and porcine viral diseases, such as hepatitis B virus (HBV), hepatitis C virus (HCV), human immunodeficiency virus (HIV), human papillomavirus (HPV), porcine reproductive and respiratory syndrome virus (PRRSV), and African swine fever virus (ASFV). However, systemic toxicity and short half-life limit the clinical application of interferon-based therapies. Therefore, developing highly effective interferon-based antiviral formulations is crucial for disease prevention and control.

Following viral invasion, host organisms rapidly upregulate interferon production, which mediates potent antiviral responses through three coordinated mechanisms: (1) engagement of cognate cell surface receptors to initiate JAK-STAT signaling cascades, (2) modulation of metabolic pathways to establish an antiviral state, and (3) transcriptional activation of interferon-stimulated genes (ISGs) encoding key effector proteins including viral restriction factors

Type I interferon (IFN) receptors consist of two subunits, IFNAR-1 and IFNAR-2, which are widely expressed on the surface of various cell types. When Type I IFNs bind to their receptors, the intracellular domains of IFNAR-1 and IFNAR-2 associate with tyrosine kinases JAK-1 and TYK2, inducing rapid phosphorylation. The phosphorylated receptor recruits signal transducers and activators of transcription (STAT) proteins, such as STAT1 and STAT2. These STAT proteins then form homodimers or heterodimers, which interact with interferon regulatory factor 9 (IRF-9) to generate the interferon-stimulated gene factor 3 (ISGF-3) complex. The ISGF-3 complex translocates to the nucleus, where it binds to interferon-stimulated response elements (ISREs) on ISG promoters, thereby activating downstream gene transcription. This pathway represents the classical JAK-STAT signaling mechanism.

Recent studies indicate that Type I interferons function beyond the classical JAK-STAT pathway, regulating broader biological processes through additional signaling pathways. For example, type I IFNs regulate cell survival and apoptosis via the PI3K/AKT pathway. In neutrophils, IFN-β activates NF-κB and PKC-δ, exerting anti-apoptotic effects. These alternative pathways provide new research directions for understanding the diverse biological functions of Type I interferons.

In addition to directly inducing an antiviral state, interferons serve as key immunoregulators, playing a crucial role in modulating immune responses. It can activate the innate immune system, by enhancing the cytotoxicity of natural killer (NK) cells to accelerate the clearance of infected cells; enhance the dendritic cell (DC) function, by promoting antigen presentation and IL-12 secretion, inducing T-cell differentiation into Th1 cells, and improving clearance of viral and intracellular bacterial infections; and boosting adaptive immunity, by stimulating T and B cells, increasing antibody production (IgG and IgA), and promoting cell-mediated immune responses.

Interferons can function as vaccine adjuvants by activating innate immune pathways, enhancing antigen recognition, and improving T and B cell responses. They optimize both the magnitude and direction of immune responses. For example, interferon combined with a classical swine fever virus (CSFV) vaccine significantly increases antibody titers and extends protection. Interferon combined with a pseudorabies virus (PRV) vaccine enhances cytotoxic T lymphocyte (CTL) activity and improves viral clearance. The integration of interferon genes into probiotic can provide long-term modulation of gut immunity and exert anti-inflammatory effects. Additionally, interferon mRNA has great potential in adjuvant applications, addressing the limitations of mRNA vaccines and enhancing their immunogenicity. For example, IFN-α mRNA combined with COVID-19 or influenza mRNA vaccines enhances neutralizing antibody levels and provides better protection against viral variants. IFN-α mRNA combined with CSFV inactivated vaccines significantly improves vaccine immunogenicity and cellular immune responses.

Currently, systematic experimental data on the antiviral activity of different porcine interferon subtypes remains lacking. Additionally, research on interferon protein modifications and mutations is still largely empirical, leading to suboptimal interferon-based antiviral therapies. As a result, current clinical applications fail to meet the demands for the prevention and treatment of various porcine viral diseases.

### SUMMARY OF THE INVENTION

To address the problems and limitations in the prior art, the present invention aims to provide a porcine interferon-like recombinant protein and its applications.

To achieve this objective, the invention employs the following technical solutions:

### First Aspect

The first aspect of the invention provides a porcine interferon-like recombinant protein, which is a protein selected from one of the following:
A1) a protein having an amino acid sequence as shown in SEQ ID NO: 1;
A2) a fusion protein obtained by linking a tag to the N-terminal and/or C-terminal of the protein having an amino acid sequence as shown in SEQ ID NO: 1;
A3) a functionally equivalent protein obtained by substituting, deleting, and/or adding one or more amino acid residues to the amino acid sequence as shown in SEQ ID NO: 1;
A4) a protein having at least 85% sequence homology to the amino acid sequence as shown in SEQ ID NO: 1, while maintaining the same function;
A5) a mutated protein with 1 to 7 amino acid residue mutations compared to the amino acid sequences of 17 porcine natural IFN-α subtypes, wherein the mutation sites are selected from P(V)4A, A42V, H58Y, E71K, G72D, A74S, and A148V of the amino acid sequence as shown in SEQ ID NO: 1;
A6) a modified protein obtained by pharmaceutically acceptable protein modifications of the protein having the amino acid sequence as shown in SEQ ID NO: 1.

Preferably, for A1), to facilitate purification, a tag can be added to the N-terminal or C-terminal of the protein having the amino acid sequence as shown in SEQ ID NO: 1. Suitable tags include Poly-Arg (RRRRR; SEQ ID NO: 3), Poly-His (HHHHHH; SEQ ID NO: 4 or HHHHHHHH; SEQ ID NO: 5), FLAG (DYKDDDDK; SEQ ID NO: 6), Strep-tagII(WSHPQFEK; SEQ ID NO: 7), and c-myc (EQKLISEEDL; SEQ ID NO: 8).

Preferably, for A5), the mutation sites are defined as follows: P(V)4A: the 4th amino acid residue, originally P or V, is mutated to A; A42V: the 42nd amino acid residue, originally A, is mutated to V; H58Y: the 58th amino acid residue, originally H, is mutated to Y; E71K: the 71st amino acid residue, originally E, is mutated to K; G72D: the 72nd amino acid residue, originally G, is mutated to D; A74S: the 74th amino acid residue, originally A, is mutated to S; and A148V: the 148th amino acid residue, originally A, is mutated to V.

Preferably, for A6), protein modifications include but are not limited to: PEGylation (polyethylene glycol modification); fusion with long-half-life proteins or peptides, such as human serum albumin (HSA), immunoglobulin Fc fragments; and XTEN polypeptides, which undergo glycosylation modifications through engineering or post-translational modifications.

The second aspect of the invention provides biological materials related to the protein of the first aspect, selected from one of the following:
B1) a nucleic acid molecule encoding the protein having an amino acid sequence as shown in SEQ ID NO: 1;
B2) an expression cassette containing the nucleic acid molecule in B1;
B3) a recombinant vector containing the nucleic acid molecule in B1 or the expression cassette in B2;
B4) a recombinant microorganism containing the nucleic acid molecule in B1, the expression cassette in B2, or the recombinant vector in B3;
B5) an mRNA molecule containing the nucleic acid molecule in B1;
B6) an adjuvant containing the nucleic acid molecule in B1;
B7) a vaccine containing the nucleic acid molecule in B1.

### PREFERRED EMBODIMENTS

1). The coding sequence of the nucleic acid molecule in B1) is provided in SEQ ID NO: 2.
2). The nucleic acid molecule may be integrated into microbial genomes (e.g., Lactic Acid Bacteria, Bifidobacterium, Bacillus subtilis) via CRISPR/Cas9 for stable expression.

The third aspect of the present invention provides the following applications for the protein provided in the first aspect or the biological material provided in the second aspect:
C1) for use in the preparation of drugs for the prevention or treatment of diseases related to porcine viral infections;
C2) for use in the preparation of products that inhibit porcine viral infections;
C3) for use in the preparation of antiviral products against porcine viruses;
C4) for use in the preparation of drugs for the prevention or treatment of diseases related to bacterial infections;
C5) for use in the preparation of products that inhibit bacterial infections;
C6) for use in the preparation of drugs for the prevention or treatment of parasitic diseases;
C7) for use in the preparation of immune adjuvants of vaccines, wherein the vaccines are used for the prevention of diseases related to porcine viral, bacterial, and/or parasitic infections;
C8) for use in feed additives or feed;
C9) for use in microecological preparations;
C10) for use in mRNA products;
C11) for use in the preparation of products for regulating and/or improving the immunity of pigs.

Preferably, in the above applications, the porcine viruses include, but are not limited to, at least one of the following: Vesicular stomatitis virus, Pseudorabies virus, Classical Swine Fever virus (CSFV), Foot-and-Mouth Disease virus (FMDV), Porcine Reproductive and Respiratory Syndrome virus (PRRSV), African Swine Fever virus (ASFV), Swine Influenza virus (SIV), Porcine Circovirus Disease virus (PCV), Porcine Adenovirus, Porcine Epidemic Diarrhea virus (PEDV), Transmissible Gastroenteritis virus (TGEV), Porcine Delta Coronavirus (PDCoV), Porcine Acute Diarrhea Syndrome Coronavirus (SADS-CoV), Swine Pox Virus, Porcine Parvovirus (PPV), Japanese Encephalitis Virus (JEV), Porcine Encephalomyocarditis Virus, Porcine Enterovirus, Porcine Alphavirus Encephalitis Virus, Porcine Orthoreovirus, Seneca Valley Virus, Swine Dysentery caused by Breda Virus, Porcine Rubulavirus, and Porcine Cytomegalovirus (PCMV).

Preferably, in the above applications, the porcine bacteria include, but are not limited to, at least one of the following: Streptococcus suis, Escherichia coli, and Salmonella spp.

Preferably, in the above applications, the porcine parasites include, but are not limited to, at least one of the following: Toxoplasma gondii, Sarcoptes scabiei, and Trichinella spiralis.

Preferably, in the above applications, the products include, but are not limited to, at least one of the following: drugs, vaccine additives, feed additives, microecological preparations, reagents, and kits.

Preferably, in C7), the porcine interferon-like recombinant protein can be used as an immune adjuvant for vaccines to enhance the immune response induced by the vaccine, increase antibody titers and the level of cellular immune responses, and shorten the time required for the immune response to take effect.

Preferably, in C11), the product for regulating and/or improving the immunity of pigs is an immunomodulator, which contains the protein provided in the first aspect or the biological material provided in the second aspect. The immunomodulator can directly activate immune cells and regulate immune responses to combat viral, bacterial, and parasitic infections while also enhancing the efficacy of vaccines.

Preferably, the nucleic acid molecules or expression cassettes mentioned in the second aspect can be integrated into the genome of microecological bacteria through genetic engineering methods, enabling the microecological bacteria to express biologically active interferon. The strains of microecological preparations include, but are not limited to, the following genera: Lactic Acid Bacteria, Bifidobacterium spp., Bacillus subtilis, Bacillus spp., Enterococcus spp., Propionibacterium spp., Yeast, and Streptomyces spp. After the microecological preparation is administered orally to pigs, it can significantly enhance the intestinal immune barrier function and inhibit pathogen infections. More preferably, the nucleic acid molecules mentioned in the second aspect can be integrated into the host bacterial genome through the CRISPR/Cas9 system and stably expressed.

Preferably, in B5) and C10), mRNA products or DNA vaccines can be used alone or in combination with mRNA vaccines or traditional vaccines for the prevention and/or treatment of porcine infectious diseases.

The fourth aspect of the present invention provides a pharmaceutical composition comprising the porcine interferon-like recombinant protein as provided in the first aspect.

Preferably, the pharmaceutical composition further comprises a pharmaceutically acceptable carrier, and the pharmaceutical composition is used for the prevention or treatment of diseases related to porcine viral, bacterial, and/or parasitic infections.

Preferably, the pharmaceutically acceptable carrier/excipient includes (but is not limited to): diluents; fillers such as lactose, sodium chloride, glucose, urea, starch, water, etc.; bulking agents such as starch, sucrose, etc.; binders such as simple syrup, glucose solution, starch solution, cellulose derivatives, alginates, gelatin, and polyvinylpyrrolidone; wetting agents such as glycerol; disintegrants such as dry starch, sodium alginate, kelp polysaccharide powder, agar powder, calcium carbonate, and sodium bicarbonate; absorption promoters such as quaternary ammonium compounds, sodium dodecyl sulfate, etc.; surfactants such as polyoxyethylene sorbitan fatty acid esters, sodium dodecyl sulfate, monoglycerides of stearic acid, cetyl alcohol, etc.; humectants such as glycerol, starch, etc.; adsorbent carriers such as starch, lactose, bentonite, silica gel, kaolin, and soap clay, etc.; lubricants such as talc, calcium and magnesium stearates, polyethylene glycol, boric acid powder, etc.

Preferably, the pharmaceutical composition further comprises a pharmaceutically acceptable encapsulating carrier.

Preferably, the pharmaceutically acceptable encapsulating carrier refers to encapsulating the drug into lipid nanoparticles, polymer nanoparticles, or other delivery systems, which can achieve a sustained-release effect, prolong its retention time in the body, and improve tissue distribution and targeting performance.

Preferably, the pharmaceutical composition can be administered by injection, spray, nasal drop, ophthalmic drop, oral, or drinking water, and can be introduced into the body through permeation, absorption, or physical or chemical mediation, such as into muscle, intradermal, subcutaneous, intravenous, mucosal tissue; or it can be introduced into the body after being mixed or encapsulated with other substances.

The fifth aspect of the present invention provides a feed additive, which comprises the porcine interferon-like recombinant protein as provided in the first aspect and/or the encoding gene of the porcine interferon-like recombinant protein as provided in the second aspect.

The sixth aspect of the present invention provides an immune adjuvant, which comprises the porcine interferon-like recombinant protein as provided in the first aspect and/or the encoding gene of the porcine interferon-like recombinant protein as provided in the second aspect.

The seventh aspect of the present invention provides an mRNA preparation, which comprises the porcine interferon-like recombinant protein as provided in the first aspect and/or the encoding gene of the porcine interferon-like recombinant protein as provided in the second aspect.

The eighth aspect of the present invention provides a method for preparing the recombinant protein as provided in the first aspect, which comprises: constructing a recombinant expression vector containing a nucleic acid molecule encoding the porcine interferon-like recombinant protein as provided in the first aspect, introducing the recombinant expression vector into a host cell to obtain recombinant cells expressing the recombinant protein, culturing the recombinant cells to express the recombinant protein, and obtaining the recombinant protein by separation and purification.

In the present invention, the term “interferon-like” refers to the functional and/or structural characteristics exhibited by interferon or interferon analogs, or the expected similar functional and/or structural characteristics. For example, “interferon-like biological activity” includes antiviral, antiproliferative, and immunomodulatory activities.

Compared with the existing technologies, the present invention has achieved the following positive and beneficial effects:

By constructing a PoIFN-α DNA shuffling bacterial library and screening the bacterial library, the present invention has obtained a porcine interferon-like recombinant protein Porferon with high activity. This recombinant protein has good antiviral effects against porcine viruses (such as porcine reproductive and respiratory syndrome virus, vesicular stomatitis virus, pseudorabies virus, porcine epidemic diarrhea virus, porcine circovirus, etc.), bacteria (such as Streptococcus suis, porcine intestinal bacteria, etc.), and parasites (such as Toxoplasma gondii, Sarcoptes scabiei, Trichinella spiralis, etc.), and its antiviral, antibacterial, and antiparasitic activities are significantly higher than those of natural porcine interferon. Therefore, the porcine interferon-like recombinant protein Porferon of the present invention can be used to prepare drugs for the prevention or treatment of diseases related to porcine viral, bacterial, or parasitic infections, providing a new preventive and/or therapeutic solution for porcine antiviral, antibacterial, and antiparasitic infections.

### BRIEF DESCRIPTION OF THE DRAWINGS

FIG. 1 shows the amino acid sequence alignment results of Porferon protein with 17 types of porcine α-interferon.

FIG. 2 presents the phylogenetic tree of the amino acid sequence alignment of Porferon protein with 17 types of porcine α-interferon.

FIG. 3 illustrates the nucleotide sequence alignment results of the Porferon protein-encoding nucleotide sequence with PoIFN-α17. Since the first three nucleotides (ATG) at the 5′ end of the Porferon protein-encoding nucleotide sequence were artificially added as the start codon, the nucleotide sequence in FIG. 3 is numbered starting from the fourth nucleotide at the 5′ end, with a total of 474 nucleotides counted.

FIG. 4 shows the amino acid sequence alignment results of Porferon protein with PoIFN-α17. In FIG. 4, when numbering the amino acids of Porferon protein, the methionine (Met) translated from the start codon added for gene expression is considered as amino acid number 0. The first amino acid following this methionine, cysteine (which is also the first amino acid of the mature protein of natural porcine interferon), is numbered as the first amino acid. Therefore, the amino acid sequence contains a total of 158 amino acid residues.

FIG. 5 displays the results of the inhibition of PRRSV replication by Porferon protein in MARC-145 cells.

FIG. 6 shows the results of the inhibition of PRRSV replication by natural PoIFN-α17 and Porferon protein in MARC-145 cells.

FIGS. 7A to 7C presents the clinical trial results in piglets, including body temperature statistics (FIG. 7A), survival rate statistics (FIG. 7B), and clinical scoring results (FIG. 7C).

FIG. 8 shows the detection results of PRRSV genome cDNA copy numbers.

FIG. 9 presents the lung lesion photographs of piglets in the virus control group and the Porferon treatment group. The left image shows the lung lesion photograph of a deceased piglet in the virus control group, while the right image shows the lung lesion photograph of a piglet in the Porferon treatment group.

### DETAILED DESCRIPTION OF THE EMBODIMENTS

To enable those skilled in the art to more clearly understand the technical solutions of the present invention, the technical solutions will be described in detail below in conjunction with specific embodiments.

### Example 1: Obtaining the Porcine Interferon-Like Recombinant Protein Porferon

### 1. Construction of the Initial Porcine Interferon-Alpha (PoIFN-α) DNA Shuffling Bacterial Library

There are 17 types of interferon α subtypes in pigs, namely: PoIFN-α1 (Gene bank accession number GQ415055), PoIFN-α2 (Gene bank accession number GQ415056), PoIFN-α3 (Gene bank accession number GQ415057), PoIFN-α4 (Gene bank accession number GQ415058), PoIFN-α5 (Gene bank accession number GQ415059), PoIFN-α6 (Gene bank accession number GQ415060), PoIFN-α7 (Gene bank accession number GQ415061), PoIFN-α8 (Gene bank accession number GQ415062), PoIFN-α9 (Gene bank accession number GQ415063), PoIFN-α10 (Gene bank accession number GQ415064), PoIFN-α11 (Gene bank accession number GQ415065), PoIFN-α12 (Gene bank accession number GQ415066), PoIFN-α13 (Gene bank accession number GQ415067), PoIFN-α14 (Gene bank accession number GQ415068), PoIFN-α15 (Gene bank accession number GQ415069), PoIFN-α16 (Gene bank accession number GQ415070), and PoIFN-α17 (Gene bank accession number GQ41507).

Based on the nucleotide sequences of the mature protein genes of these 17 PoIFN-α subtypes, 15 pairs of degenerate oligonucleotide fragments were designed and synthesized as primers. These primers encompass all DNA sequences of natural porcine interferon-alpha subtypes. The specific nucleotide sequences of the forward oligonucleotide fragments from these pairs are as follows (only the forward sequences are listed, and the reverse complementary sequences are not included):

Frag1:
(SEQ ID NO: 9)
5′-CGAAGCATTGGTTAAAAATTAAGGAGGAAGGATCC ATGGCCCCA
(A/T)CCTCA(G/A)C(C/T)(T/C)TC(C/T)TCA(C/T)(A/G)G
(C/T)C CTGGTGCTAC-3′;
Frag2:
(SEQ ID NO: 10)
5′-C(C/T)TCA(C/T)(A/G)G(C/T)CCTGGTGCTAC TCAGCT(G
/A)CAA(T/G)GCCATCT(G/A)CT(G/C)TCTGGGCT GC(G/A)A
CC(T/C)G(C/G)(C/T)-3′;
Frag3:
(SEQ ID NO: 11)
5′-T(G/C)TCTGGGCTGC(G/A)ACC(T/C)G(C/G)(C/T) TCAG
ACC(C/T)AC AGCCTGGCTC ACACCAGGGC CCTGAGGC(T/C)C-
3′;
Frag4:
(SEQ ID NO: 12)
5′-ACACCAGGGCCCTGAGGC(T/C)CCTGGCACAAA TGAGGAGAAT
CTCCCCCTTC TCCTGCCTGG-3′;
Frag5:
(SEQ ID NO: 13)
5′-CTCCCCCTTCTCCTGCCTGGACCACAGAA(G/A) GGACTTTGGA
T(T/C)CCC(C/T)CA(A/T)G AGGC(C/T)TT(G/T)GG-3′;
Frag6:
(SEQ ID NO: 14)
5′-T(T/C)CCC(C/T)CA(A/T)GAGGC(C/T)TT(G/T)GG GGGC
AACCAG GTCCAGAAGG CTCAAGCCAT GGCTCTGGTG-3′;
Frag7:
(SEQ ID NO: 15)
5′-CTCAAGCCAT GGCTCTGGTG CATGAGATGC TCCAGCAGAC C
T(T/C)CCAGCTC TTCAGCACAG-3′;
Frag8:
(SEQ ID NO: 16)
5′-CT(T/C)CCAGCTCTTCAGCACAGAGGGCTC(G/A)GC TGCTGC
CTGG (G/A)ATGA(G/C)AGCC TCCTGCACCA-3′;
Frag9:
(SEQ ID NO: 17)
5′-(G/A)ATGA(G/C)AGCC TCCTGCACCA GTTCTGCACT GGAC
TGGATC AGCAGCTCAG GGACCTGGAA-3′;
Frag10:
(SEQ ID NO: 18)
5′-AGCAGCTCAG GGACCTGGAA GCCTGTGTCA T(G/A)CAGGAG
G(C/T) (G/C)GGGCTGGAA GGGAC(C/G)CCCC-3′;
Frag11:
(SEQ ID NO: 19)
5′-(G/C)GGGCTGGAA GGGAC(C/G)CCCC TGCTGGAGGA GG(A
/G)CTCCATC CTGGCTGTGA GGAAATACTT-3′;
Frag12:
(SEQ ID NO: 20)
5′-CTGGCTGTGA GGAAATACTT CCACAGACTC A(C/T)CCTCTA
TC TGCA(A/G)GAGAA GA(G/A)CTACAGC-3′;
Frag13:
(SEQ ID NO: 21)
5′-TGCA(A/G)GAGAA GA(G/A)CTACAGC CCTCTGTGCCT GGG
AGATC(G/A)T CAGGGCAGAA GTCATGA(G/C)A(G/T)-3′;
Frag14:
(SEQ ID NO: 22)
5′-CAGGGCAGAA GTCATGA(G/C)A(G/T) CTCTTCTCTTC CTC
CA(C/G)A(A/C)AC CTGCAAGAC A GACTCAGGA(A/G)-3′;
and
Frag15:
(SEQ ID NO: 23)
5′-CTGCAAGACA GACTCAGGA(A/G) GAAGGAG CTCGAG CAC
CAC CAT CAC CAT CAC CAT CAC TAA GT GAATTCTGCA
GATATCCATC-3′.

In Frag1, a BamHI restriction enzyme site and the ATG start codon for protein translation are included. In Frag5, codons encoding eight consecutive histidine (His) residues, a TAA stop codon, and an EcoRI restriction enzyme site are present.

The 15 pairs of oligonucleotide fragments were mixed in equimolar amounts and assembled via PCR recombination following the method described in Stemmer W P. (1994) DNA shuffling by random fragmentation and reassembly: in vitro recombination for molecular evolution. Proc Natl Acad Sci USA, 91(22):10747-10751. The resulting PCR product was digested with the BamHI and EcoRI restriction enzymes and cloned into the E. coli expression vector pCMH. The pCMH expression vector is a derivative of pBV220, containing BamHI and EcoRI sites in its multiple cloning regions, as described in Zhang Z Q et al. (1990) Construction and application of a high-level expression vector containing PRPL promoter. Chinese J of Virol, 2:18-23. After constructing the expression plasmid library, DNA sequence analysis (PE Applied Biosystems, USA) was performed to verify sequence accuracy and recombination efficiency. The verified plasmids were then transformed into competent E. coli DH5a cells, forming the initial porcine interferon-alpha (PoIFN-α) DNA shuffling library.

During PCR amplification of genes using the primers above, a conventional DNA polymerase (New England Biolab, MA, USA) was used instead of a high-fidelity DNA polymerase.

The initial PoIFN-α DNA shuffling library was transformed into E. coli DH5a using standard methods, generating the initial bacterial library for PoIFN-α DNA shuffling.

2. Construction of the Secondary E. coli Library for Porcine Interferon-Alpha (PoIFN-α) DNA Shuffling

The bacterial DNA from the initial library or selected bacterial clones from the secondary library (see Step 3) was amplified using the first pair of PCR primers CMHF3: 5′-ACCATGAAGGTGACGCTC-3′ (SEQ ID NO: 24) and CMHR3: 5′-AATCTTCTCTCATCCGC-3′ (SEQ ID NO: 25). These primers correspond to the upstream and downstream flanking sequences of the multiple cloning site of the pCMH plasmid vector. The resulting PCR product was digested with DNase I, followed by primerless PCR assembly. The assembled fragments were then amplified again using the second pair of primers CMHF1: 5′-GAAGGCTTTGGGGTGTGTGATA-3′ (SEQ ID NO: 26) and CMHR1: 5′-TGGCGGATGAGAGAAGAT-3′ (SEQ ID NO: 27). After amplification, the PCR product was digested with BamHI and EcoRI and cloned into the pCMH expression vector to construct a plasmid library. The recombinant plasmids were then transformed into competent E. coli DH5a cells. Subsequent DNA sequencing analysis (PE Applied Biosystems, USA) was performed to confirm sequence accuracy and recombination efficiency, thereby generating the secondary bacterial library for PoIFN-α DNA shuffling.

### 3. Screening of the DNA Shuffling Bacterial Library

Freshly transformed E. coli DH5a cells were plated on LB agar and incubated overnight at 37° C. Individual bacterial colonies were picked and inoculated into 100 L LB medium containing 50 g/mL ampicillin in a 96-well plate. The cultures were incubated at 30° C. with shaking at 250 rpm overnight. After incubation, 10 L of each bacterial culture was transferred into two separate 96-well plates containing 100 L LB medium with 50 g/mL ampicillin. One plate was used as the stock plate, stored at 4° C. The other plate was incubated at 30° C. until OD600 reached 0.4, then heat-induced at 42° C. for 4 hours. After induction, the bacterial culture was immediately frozen at −80° C. for three freeze-thaw cycles. The bacterial suspension/lysate was then diluted to the required concentration and tested for antiviral activity using the PK15-VSV system. Positive clones were identified following the antiviral activity assay described in Cheng et al. (2006), Gene 382: 28-38.

On day 1, PK-15 cells (purchased from the National Institutes for Food and Drug Control) were seeded at a density of 14,000 cells per well in a 96-well plate and incubated at 37° C. On day 2, 2-fold serial dilutions of recombinant bacterial lysate, PoIFN-α17, or blank medium were added to each well and incubated at 37° C. for another 24 hours. On day 3, the medium was removed and replaced with a medium containing 1,000 PFU of vesicular stomatitis virus (VSV, ATCC, catalog number VR-1421). Cells were incubated at 37° C. for 24 hours, then examined under a microscope for cytopathic effects (CPE). Bacterial clones that inhibited CPE by more than 50% were considered positive clones.

Initially, bacterial lysate of 100 clones were serially diluted to determine the working dilution at which only 1-2% of the bacterial clones could inhibit more than 50% of the cells from showing lesions. The bacterial lysate was diluted to this working dilution for antiviral activity testing. If the working dilution was found to be too high or too low, adjustments were made accordingly.

A total of over 50,000 bacterial colonies were screened, and approximately 100 clones with the highest antiproliferative or antiviral activity were selected for further validation. The preliminary validation steps: Selected bacterial cultures were streaked on LB agar plates with 50 g/mL ampicillin and incubated overnight at 37° C. Then a single colony was picked and inoculated into 1 mL LB medium with 50 g/mL ampicillin in a test tube. The culture was incubated overnight at 30° C. with shaking at 250 rpm. 40 L of cultured bacteria was transferred into 1 mL fresh LB medium with 50 g/mL ampicillin and incubated at 30° C. until OD600 reached 0.4. Then heat induction was performed at 42° C. for 4 hours, followed by freeze-thaw cycles. The bacterial suspension/lysis product was diluted to the desired concentration and tested for antiviral activity in the PK15-VSV system to screen for positive clones.

After preliminary validation, approximately 30 colonies exhibiting the highest antiviral activity were selected. The plasmids and their insert fragments were then subjected to automated sequencing. Clones containing unique DNA sequences of the insert fragment pCMH were identified, and the corresponding PCR amplification products were used to construct the next round (secondary) DNA shuffling library.

The process of “construction of the secondary DNA shuffling library and screening of the bacterial library” was repeated. With each additional screening round, the rate of increase in antiviral activity among selected mutants was gradually declined, while the sequence homology among the selected mutants was progressively increased. By the fourth round, preliminary validation indicated that the selected mutants exhibited comparable antiviral activity, and further DNA sequencing confirmed a high degree of homology among them.

From the fourth screening round, eight colonies with high antiviral activity were selected and inoculated into 4 mL of LB medium containing 50 μg/mL ampicillin, followed by incubation at 37° C. for 16-18 hours. The overnight culture was then diluted 1:100 into a large-volume LB medium supplemented with 50 μg/mL ampicillin and incubated at 30° C. with shaking at 220 rpm. When the culture reached mid-logarithmic growth (OD600=0.5-0.6), the temperature was rapidly increased to 42° C., and shaking at 220 rpm was continued to induce Porferon expression.

Following 4 hours of heat induction, the bacterial culture was centrifuged at 5,000 rpm for 10 minutes, and the pellet was washed three times with PBS (137 mM NaCl, 2.7 mM KCl, 10 mM Na₂HPO₄, 2 mM KH₂PO₄, pH 7.4). The bacterial pellet was then resuspended in 20 mM Tris-HCl (pH 8.0) and sonicated at 300 W using a cycle of 5 seconds of sonication followed by 5 seconds of rest for 25 minutes, until the suspension became clear. After centrifugation, the supernatant was purified using an AKTA purification system, employing a Cytiva HiTrap Capto Q ion exchange column (Catalog No.: 29400462) and a Cytiva (GE Life) HiLoad 16/600 Superdex 75 pg gel filtration column (Catalog No.: 28989333).

Initially, the supernatant was loaded onto the ion exchange column and eluted using a gradient buffer (20 mM Tris-HCl, 100 mM-1 M NaCl, pH 8.0). The target protein was collected and further purified via gel filtration. The protein fraction purified via ion exchange chromatography was then loaded onto the HiLoad 16/600 Superdex 75 pg gel filtration column and eluted using PBS buffer (137 mM NaCl, 2.7 mM KCl, 10 mM Na₂HPO₄, 2 mM KH₂PO₄, pH 7.4). The target protein was collected based on real-time UV monitoring.

After antiviral activity testing using the PK15-VSV system, one mutant with the highest antiviral activity was selected from the eight mutant proteins and named Porferon. The amino acid sequence of Porferon is provided as SEQ ID NO: 1, and the corresponding nucleotide sequence is shown in SEQ ID NO: 2.

SEQ ID NO: 1:
MCNLAQTHSLAHTRALRLLAQMRRISPFSCLDHRRDFGFPQEVLG
GNQVQKAQAMALVYEMLQQTFQLFSTKDSSAAWDESLLHQFCTGL
DQQLRDLEACVMQEVGLEGTPLLEEDSILAVRKYFHRLTLYLQEK
SYSPCAWEIVRVEVMRAFSSSRNL.
SEQ ID NO: 2:
ATGTGCAACCTGGCTCAGACCCACAGCCTGGCTCACACCCGTGCG
CTGCGTCTGCTGGCGCAGATGCGTCGTATCAGCCCGTTCAGCTGC
CTGGATCACCGTCGTGATTTCGGCTTCCCGCAGGAAGTGCTGGGC
GGTAACCAGGTTCAGAAAGCGCAGGCGATGGCGCTGGTTTACGAA
ATGCTGCAGCAGACCTTCCAGCTCTTCAGCACAAAGGACTCATCT
GCTGCCTGGGATGAGAGCCTCCTGCACCAGTTCTGCACTGGACTG
GATCAGCAGCTGCGTGATCTGGAAGCGTGCGTTATGCAGGAAGTT
GGCCTGGAAGGTACCCCGCTGCTGGAAGAAGATAGCATCCTGGCG
GTTCGTAAATACTTCCACCGTCTGACCCTGTACCTGCAGGAAAAA
TCTTACAGCCCGTGCGCGTGGGAAATCGTTCGTGTGGAAGTTATG
CGTGCGTTCAGCAGCAGCCGTAACCTG

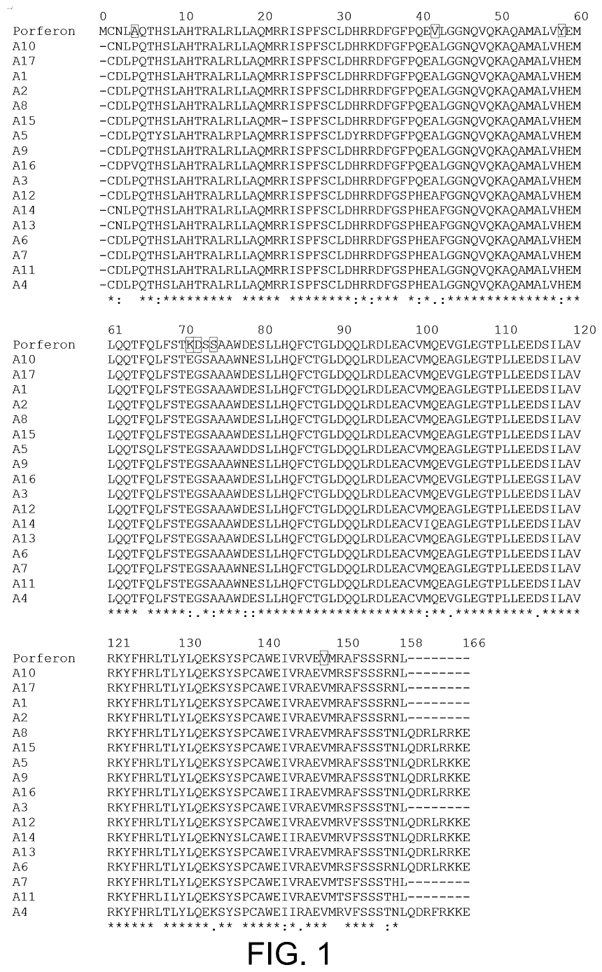

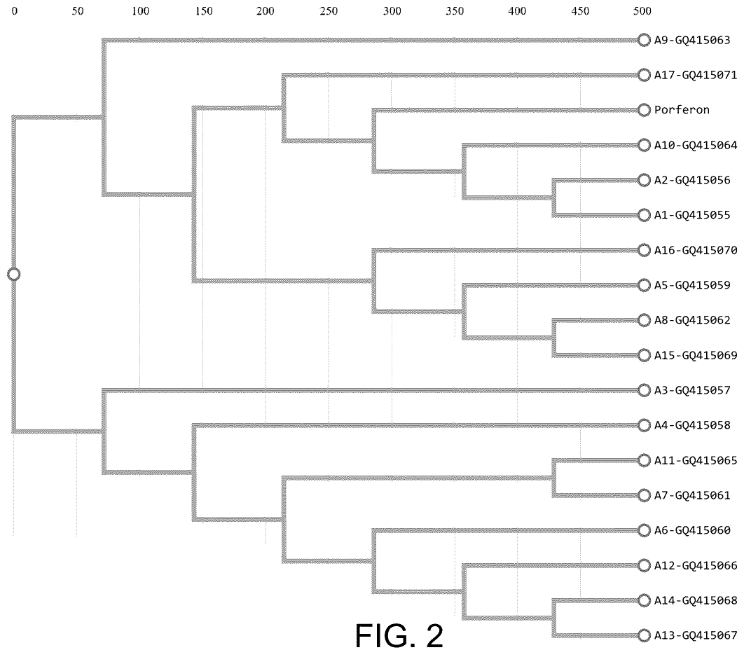

sequence alignment was conducted to compare the amino acid homology between Porferon and the 17 natural porcine α-interferon subtypes. The alignment results are presented in FIG. 1. Based on these results, a phylogenetic tree was constructed, as shown in FIG. 2.

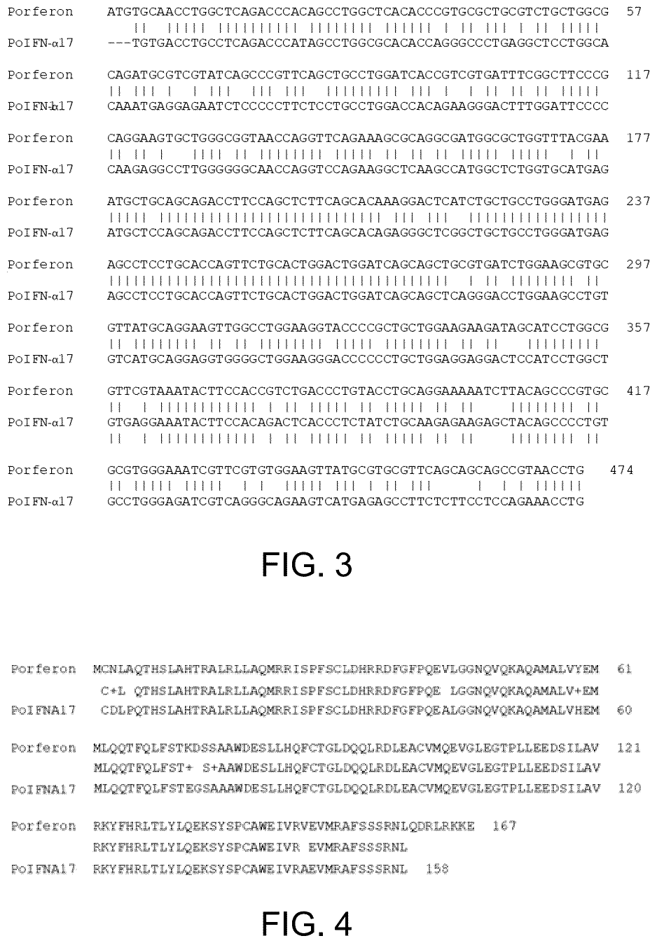

As depicted in FIGS. 1 and 2, Porferon exhibits the highest sequence homology with PoIFN-α17 at both the nucleotide and amino acid levels. The BLAST alignment results comparing the coding nucleotide sequence and amino acid sequence of Porferon with PoIFN-α17 are displayed in FIGS. 3 and 4, respectively. As shown in FIGS. 3 and 4, the Porferon coding nucleotide sequence shares 77.4% homology (367/474) with PoIFN-α17; and the Porferon amino acid sequence shares 95% homology (150/158) with PoIFN-α17.

### Example 2: Expression and Purification of Porferon Protein

The recombinant clones expressing Porferon protein, selected during the fourth round of screening in Example 1, were re-streaked on LB agar plates. Single colonies were picked and inoculated into 4 mL of LB medium containing 50 g/mL ampicillin, followed by incubation at 37° C. for 16-18 hours. The overnight culture was then diluted 1:100 into a large-volume LB medium supplemented with 50 g/mL ampicillin and incubated at 30° C. with shaking at 220 rpm. When the culture reached the mid-logarithmic growth phase (OD600=0.5-0.6), the temperature was rapidly increased to 42° C., and shaking at 220 rpm was continued for 4 hours to induce Porferon expression.

Following 4 hours of heat induction, the bacterial culture was centrifuged at 5,000 rpm for 10 minutes, and the pellet was washed three times with PBS (137 mM NaCl, 2.7 mM KCl, 10 mM Na₂HPO₄, 2 mM KH₂PO₄, pH 7.4). The bacterial pellet was resuspended in 20 mM Tris-HCl (pH 8.0) and sonicated at 300 W, using 5-second pulses followed by 5-second intervals for a total of 25 minutes, until the suspension became clear.

After centrifugation, the supernatant was purified using an AKTA purification system, employing a Cytiva HiTrap Capto Q ion exchange column (Catalog No.: 29400462) and a Cytiva (GE Life) HiLoad 16/600 Superdex 75 pg gel filtration column (Catalog No.: 28989333). The supernatant was first loaded onto the ion exchange column and eluted using a gradient buffer (20 mM Tris-HCl, pH 8.0, 100 mM-1 M NaCl). The target protein was collected and further purified using gel filtration chromatography. The purified fraction was then loaded onto the HiLoad 16/600 Superdex 75 pg gel filtration column and eluted with PBS buffer (137 mM NaCl, 2.7 mM KCl, 10 mM Na₂HPO₄, 2 mM KH₂PO₄, pH 7.4). The target protein was collected based on real-time UV monitoring, yielding Porferon protein. SDS-PAGE analysis (15%) confirmed that Porferon appeared as a single band with a molecular weight (MW) of 18-19 kDa.

To facilitate a direct comparison between Porferon and PoIFN-α17, PoIFN-α17 was also expressed and purified in Escherichia coli.

The expression plasmid pCMHPoIFNa17 carries the DNA coding sequence for mature PoIFN-α17 (GeneBank Accession No.: GQ415071), with an artificial ATG start codon added at the 5′ end. The expression of PoIFN-α17 was performed according to the protocol described by Erik Remaut, Patrick Stanssens and Walter Fiers (1983) “Inducible high-level synthesis of mature human fibroblast interferon in Escherichia coli.” Nucleic Acids Research, 11(14). The pCMHPoIFNa17 expression plasmid was transformed into E. coli DH5a cells. Single colonies were picked and inoculated into 4 mL LB medium containing 50 g/mL ampicillin, followed by incubation at 30° C. for 8 hours. Subsequently, 2 mL of the cultured bacteria was transferred into 50 mL LB medium with 50 g/mL ampicillin and incubated at 30° C. for 16-18 hours.

The next morning, the bacterial culture was diluted 1:100 into a large-volume LB medium containing 50 g/mL ampicillin and incubated at 30° C. with shaking. Once the culture reached the mid-logarithmic growth phase (OD550=0.5-0.6), the temperature was rapidly raised to 42° C. for 4 hours to induce PoIFN-α17 expression.

Following 4 hours of heat induction, the bacterial culture was centrifuged at 5,000 rpm for 10 minutes, and the pellet was washed three times with PBS buffer (137 mM NaCl, 2.7 mM KCl, 10 mM Na₂HPO₄, 2 mM KH₂PO₄, pH 7.4). The bacterial pellet was resuspended in 20 mM Tris-HCl (pH 8.0) and sonicated at 300 W using a 5-second pulse/rest cycle for 25 minutes, until the suspension became clear. The supernatant was purified using an AKTA purification system, employing a Cytiva HiTrap Capto Q ion exchange column (Catalog No.: 29400462) and a Cytiva (GE Life) HiLoad 16/600 Superdex 75 pg gel filtration column (Batch No.: 28989333). The supernatant was loaded onto the ion exchange column, eluted with a gradient buffer (20 mM Tris-HCl, 100 mM-1 M NaCl, pH 8.0), and further purified using gel filtration chromatography.

The purified PoIFN-α17 protein was analyzed by 15% SDS-PAGE, confirming a single band with a molecular weight (MW) of 18-19 kDa.

### Example 3: Tittering the Anti-VSV Activity of Porferon

The antiviral activity was determined using the PK15-VSV system according to the method of Cheng et al. (Cheng G, Chen W, Li Z, Yan W, Zhao X, Xie J, Liu M, Zhang H, Zhong Y, Zheng Z. Characterization of the porcine alpha interferon multigene family. Gene 382: 28-38, 2006). The specific operations were as follows: on the first day, PK15 cells (purchased from the National Institutes for Food and Drug Control of China) were seeded at a density of 14,000 cells per well in a 96-well plate and incubated at 37° C.; on the second day, 2-fold serial dilutions of Porferon or PoIFN-α17 with an original concentration of 0.5 μg/mL were added to each well, respectively, and the medium without Porferon or PoIFN-α17 was used as the blank control, and incubated again at 37° C. for 24 hours; on the third day, the medium was removed and replaced with the medium containing 1,000 PFU of vesicular stomatitis virus (VSV, ATCC, catalog number VR-1421). The cells were incubated again at 37° C. for 24 hours, and the results were observed under a microscope, first observing whether there were cytopathic effects (CPE) in the “cell control wells” and “virus control wells”.

The antiviral activity was determined using the PK15-VSV system following the methodology of Cheng et al. (Gene 382:28-38, 2006). The specific experimental steps were as follows: Day 1: PK15 cells (obtained from the National Institutes for Food and Drug Control, China) were seeded into a 96-well plate at a density of 14,000 cells per well and incubated at 37° C. Day 2: Serial two-fold dilutions of Porferon or PoIFN-α17 (initial concentration: 0.5 μg/mL) were added to designated wells. Wells containing medium without Porferon or PoIFN-α17 served as the blank control. The plate was incubated at 37° C. for 24 hours. Day 3: The medium was replaced with fresh medium containing 1,000 plaque-forming units (PFU) of vesicular stomatitis virus (VSV; ATCC VR-1421). After 24 hours of incubation at 37° C., cytopathic effects (CPE) were evaluated microscopically. The “cell control wells” (untreated cells) and “virus control wells” (cells infected with VSV only) were first examined to validate assay conditions.

The antiviral titer was defined as the reciprocal of the highest dilution capable of inhibiting 50% of CPE, with one titer unit representing the highest dilution (per milliliter) that protected 50% of cells from viral damage. Calculations utilized the Reed-Muench formula from the Chinese Veterinary Pharmacopoeia (2020 Edition, Part 3, Appendix):

$${Log}_{2} X ( logarithm of the 50 \% inhibitory dilution ) = {Log}_{2} ( dilution showing \geq 50 \% protection ) + ( distance ratio \times {Log}_{2} ( dilution factor ) ) .$$

Triplicate experiments were performed, with Porferon and PoIFN-α17 tested in parallel on the same 96-well plate. the statistical results of the Porferon titer determination are shown in Table 1, and the statistical results of the PoIFN-α17 titer determination are shown in Table 2.

TABLE 1
Statistical results of Porferon titer determination.

| | | | Cumulative Results | | Percentage of |
|---|---|---|---|---|---|
| Porferon | Observation Results | | Cumulative | Cumulative | Cumulative |
| Concentration | CPE (−) | CPE(+) | Inhibition | CPE | Inhibition |
| 2⁻² | 4 | 0 | 35 | 0 | 100% (35/35) |
| 2⁻³ | 4 | 0 | 31 | 0 | 100% (31/31) |
| 2⁻⁴ | 4 | 0 | 27 | 0 | 100% (27/27) |
| 2⁻⁵ | 4 | 0 | 23 | 0 | 100% (23/23) |
| 2⁻⁶ | 4 | 0 | 19 | 0 | 100% (19/19) |
| 2⁻⁷ | 4 | 0 | 15 | 0 | 100% (15/15) |
| 2⁻⁸ | 4 | 0 | 11 | 0 | 100% (11/11) |
| 2⁻⁹ | 4 | 0 | 7 | 0 | 100% (7/7) |
| 2⁻¹⁰ | 3 | 1 | 3 | 1 | 100% (3/4) |
| 2⁻¹¹ | 0 | 4 | 0 | 5 | 100% (0/5) |

TABLE 2
Statistical results of PoIFN-α17 titer determination.

| | | | Cumulative Results | | Percentage of |
|---|---|---|---|---|---|
| PoIFN-α17 | Observation Results | | Cumulative | Cumulative | Cumulative |
| Concentration | CPE(−) | CPE(+) | Inhibition | CPE | Inhibition |
| 2⁻² | 4 | 0 | 7 | 0 | 100% (5/5) |
| 2⁻³ | 3 | 1 | 3 | 1 | 75% (3/4) |
| 2⁻⁴ | 0 | 4 | 0 | 5 | 0% (0/5) |
| 2⁻⁵ | 0 | 4 | 0 | 9 | 0% (0/9) |
| 2⁻⁶ | 0 | 4 | 0 | 13 | 0% (0/13) |
| 2⁻⁷ | 0 | 4 | 0 | 17 | 0% (0/17) |
| 2⁻⁸ | 0 | 4 | 0 | 21 | 0% (0/21) |
| 2⁻⁹ | 0 | 4 | 0 | 25 | 0% (0/25) |
| 2⁻¹⁰ | 0 | 4 | 0 | 29 | 0% (0/29) |
| 2⁻¹¹ | 0 | 4 | 0 | 33 | 0% (0/33) |

Table 1 shows that the logarithm (Log₂X) of the Porferon dilution required to inhibit 50% of cell lesions is 10+0.33. This means that when 0.5 μg/mL of Porferon is diluted 2^{10.33} (approximately 1,311-fold), it can protect half of the cells from damage.

Similarly, Table 2 shows that the logarithm (Log₂X) of the PoIFN-α17 dilution required to inhibit 50% of cell lesions is 3+0.33. This indicates that when 0.5 μg/mL of PoIFN-α17 is diluted 2^{3.33} (approximately 10-fold), it can protect half of the cells from damage.

These results demonstrate that 0.25 ng/mL of purified Porferon protein can protect half of the PK-15 cells from VSV infection, corresponding to a half-maximal protective dose (PD₅₀) of 0.39 ng/mL. In contrast, the PD₅₀ of PoIFN-α17 is 50.8 ng/mL. These data indicate that the antiviral activity of Porferon is 130 times higher than that of PoIFN-α17.

### Example 4: Tittering the Activity of Porferon Against the PRRSV Propagation

MARC-145 cells were seeded into 24-well plates at a density of 5×10⁵ cells per well. Once the cells reached monolayer confluence, 200 μL of Porferon-containing culture medium at final concentrations of 0.1 ng/mL, 1 ng/mL, and 10 ng/mL was added to the cells. A control group with Porferon-free culture medium was included.

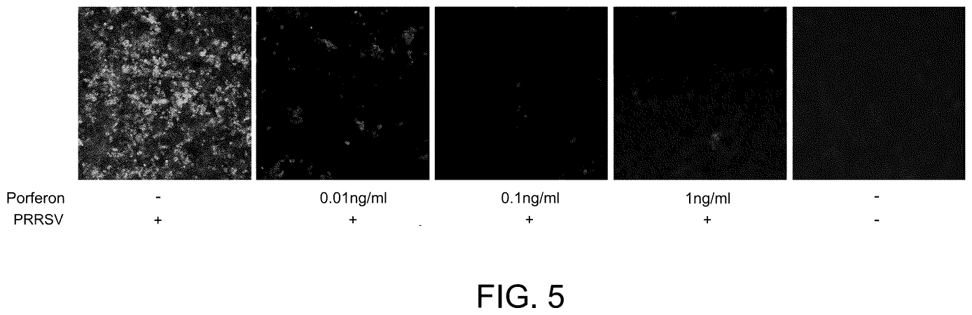

After 24 hours of incubation, the cells were infected with 1,000 TCID₅₀ of porcine reproductive and respiratory syndrome virus (PRRSV). Following a 48-hour infection period, cells were harvested, and indirect immunofluorescence assay (IFA) was used to evaluate the antiviral activity of Porferon against PRRSV. The results are shown in FIG. 5. As observed in FIG. 5, 0.01 ng/mL Porferon effectively inhibited PRRSV replication.

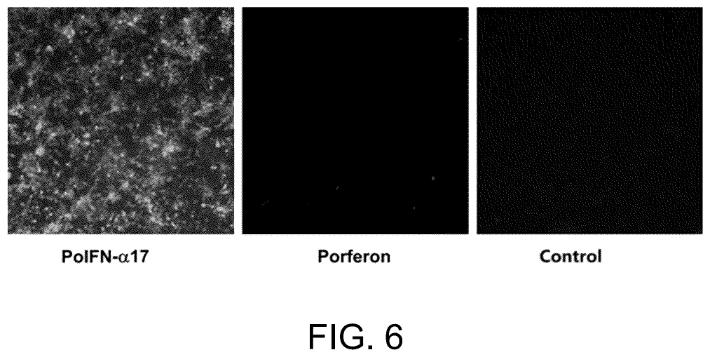

To further compare the anti-PRRSV activity of PoIFN-α17 and Porferon, MARC-145 cells were seeded into 24-well plates at 5×10⁵ cells per well. Once a monolayer was established, the culture medium was replaced with fresh medium containing 0.1 ng/mL of PoIFN-α17 or Porferon, while control wells received no PoIFN-α17 or Porferon. After 24 hours of incubation, the cells were infected with 1,000 TCID₅₀ of PRRSV. Following 48 hours of infection, the cells were harvested and analyzed using IFA to assess antiviral activity. The results are shown in FIG. 6.

As seen in FIG. 6, at a concentration of 0.1 ng/mL, no specific fluorescence was observed in the Porferon-treated wells, indicating strong inhibition of PRRSV replication. In contrast, under the same conditions, 85% of cells in the PoIFN-α17-treated wells exhibited specific fluorescence, suggesting that Porferon has significantly higher inhibitory activity against PRRSV replication compared to PoIFN-α17.

### Example 5: Antiviral Activity of Porferon Protein Against PRV

PK-15 cells were seeded into 96-well plates at a density of 14,000 cells per well and incubated at 37° C. with 5% CO₂ until a monolayer was formed. The culture medium was then replaced with fresh medium containing PoIFN-α17 or Porferon at final concentrations of: 1 μg/mL, 1×10⁻¹ g/mL, 1×10⁻² g/mL, 1×10⁻³ g/mL, 1×10⁻⁴ g/mL, 1×10⁻⁵ g/mL, 1×10⁻⁶ g/mL, 1×10⁻⁷ g/mL, 1×10⁻⁸ g/mL, 1×10⁻⁹ g/mL, 1×10⁻¹⁰ g/mL. Each dilution was tested in triplicate, with two additional control groups: Blank control (no PoIFN-α17 or Porferon) and Virus control (PRV infection without treatment). Cells were incubated at 37° C. with 5% CO₂ for 24 hours. The medium was then removed, and cells were infected with 100 TCID₅₀ of pseudorabies virus (PRV). Following 48 hours of incubation at 37° C. with 5% CO₂, cells were examined under a microscope to assess cytopathic effects (CPE). For the experiment to be considered valid, the virus control wells had to exhibit severe CPE (+++ or ++++), with 75-100% of cells displaying marked lesions, while the blank control wells needed to show healthy cell growth.

After 24 hours of pre-incubation with PoIFN-α17 or Porferon, followed by PRV infection (100 TCID₅₀) for 48 hours, CPE was evaluated, and the results are summarized in Table 3.

TABLE 3
Comparative Antiviral Activity of PoIFN-α17 and Porferon Against PRV.

| | Dose (μg/ml) | | | | | | | | | | | |
|---|---|---|---|---|---|---|---|---|---|---|---|---|
| | | | | | | | | | | | | Virus |
| | 1 | 10⁻¹ | 10⁻² | 10⁻³ | 10⁻⁴ | 10⁻⁵ | 10⁻⁶ | 10⁻⁷ | 10⁻⁸ | 10⁻⁹ | 10⁻¹⁰ | control |
| PoIF-α17 | + | ++ | +++ | +++ | ++++ | ++++ | ++++ | ++++ | ++++ | ++++ | ++++ | ++++ |
| Porferon | − | − | − | − | + | ++ | ++ | +++ | +++ | ++++ | ++++ | ++++ |

Note:
++++: 100% CPE;
+++: 75% CPE;
++: 50% CPE;
+: 25% CPE;
−: no CPE

As shown in Table 3, a concentration of 0.1 g/mL of PoIFN-α17 in the cell culture fluid is required to prevent 50% of the cells from exhibiting cytopathic effects (CPE). In comparison, only 1 to 10 pg/mL of Porferon is needed to achieve the same level of protection. This demonstrates that Porferon exhibits approximately 10,000-fold greater antiproliferative activity against PRV in PK15 cells than PoIFN-α17.

### Example 6: Animal Experiment on the Protective Effect of Porferon Against PRRSV Infection

A total of 10 healthy piglets, aged 4-5 weeks and confirmed negative for PRRSV, PCV, PEDV, and ASFV, were randomly divided into two groups: Virus Control Group and Porferon Treatment Group. All piglets were infected with PRRSV (BB0907 strain, kindly provided by the College of Veterinary Medicine, Nanjing Agricultural University) by intramuscular injection of 2 mL (1×10^{5.5} TCID₅₀) and intranasal administration of 2 mL.

Virus Control Group:
1. Intramuscular injection of 2 mL sterile PBS at the time of viral challenge.
2. Intramuscular injection of 2 mL sterile PBS 24 hours post-infection.
3. Intramuscular injection of 2 mL sterile PBS 96 hours post-infection.

Porferon Treatment Group:
1. Intramuscular injection of 8 g Porferon (2 mL) at the time of viral challenge.
2. Intramuscular injection of 8 g Porferon (2 mL) 24 hours post-infection.
3. Intramuscular injection of 8 g Porferon (2 mL) 96 hours post-infection.

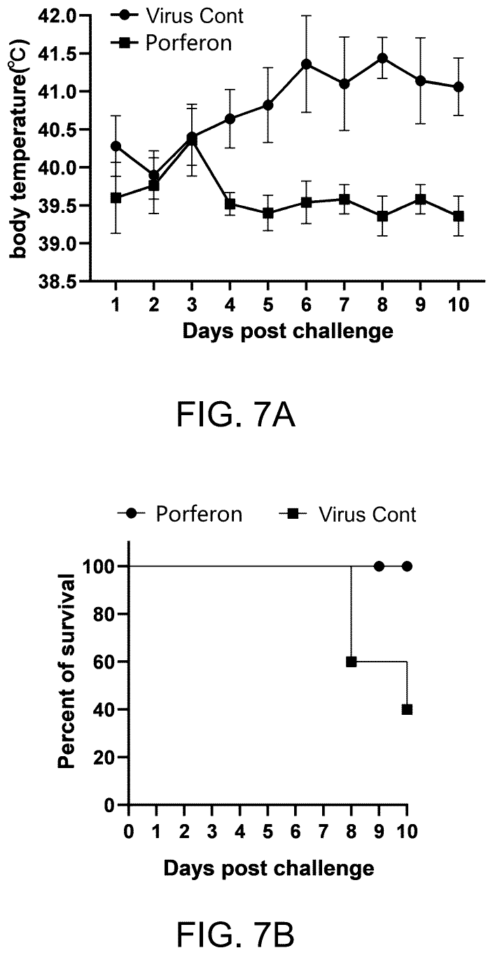

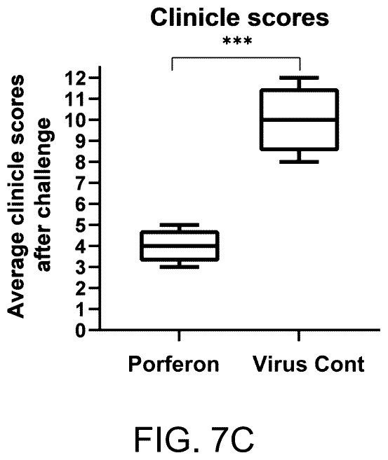

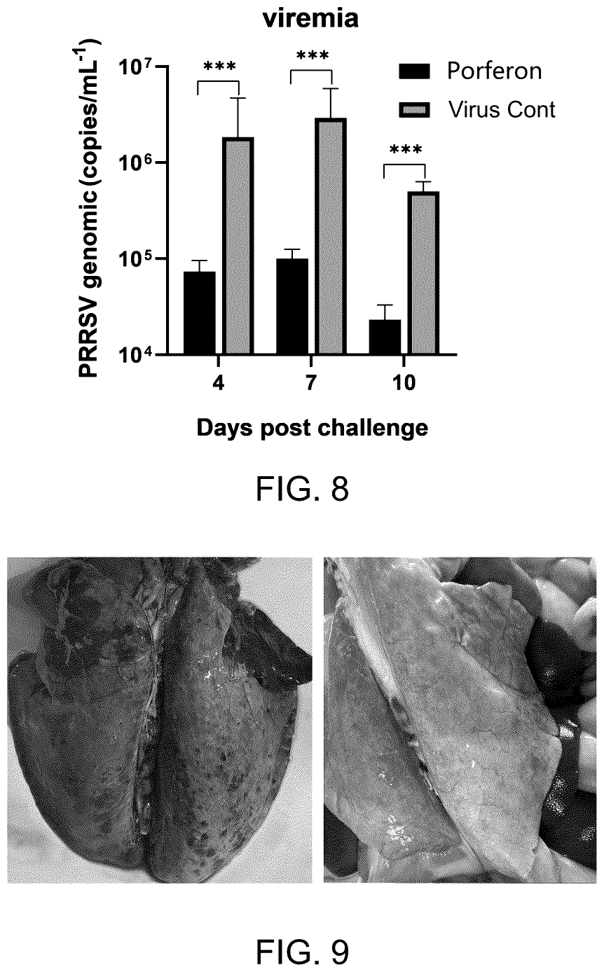

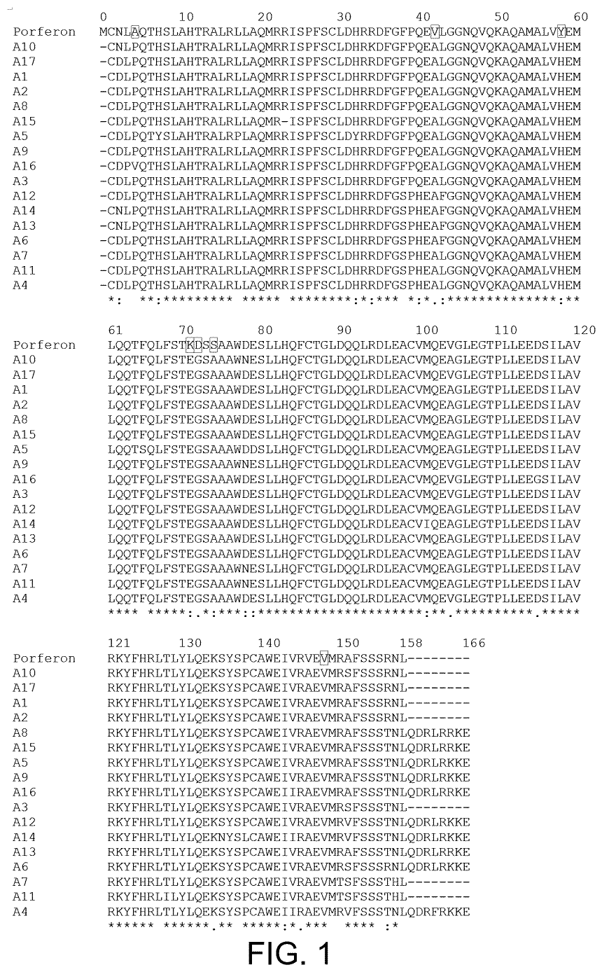

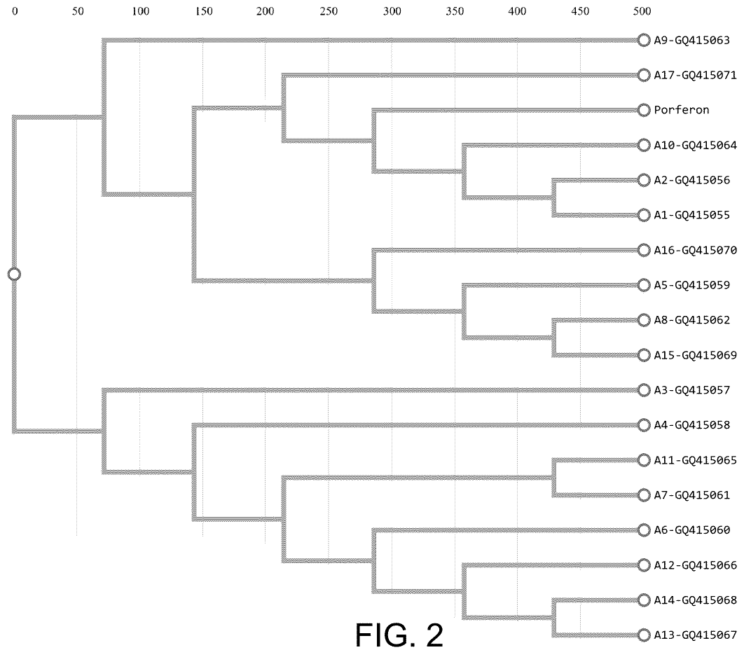

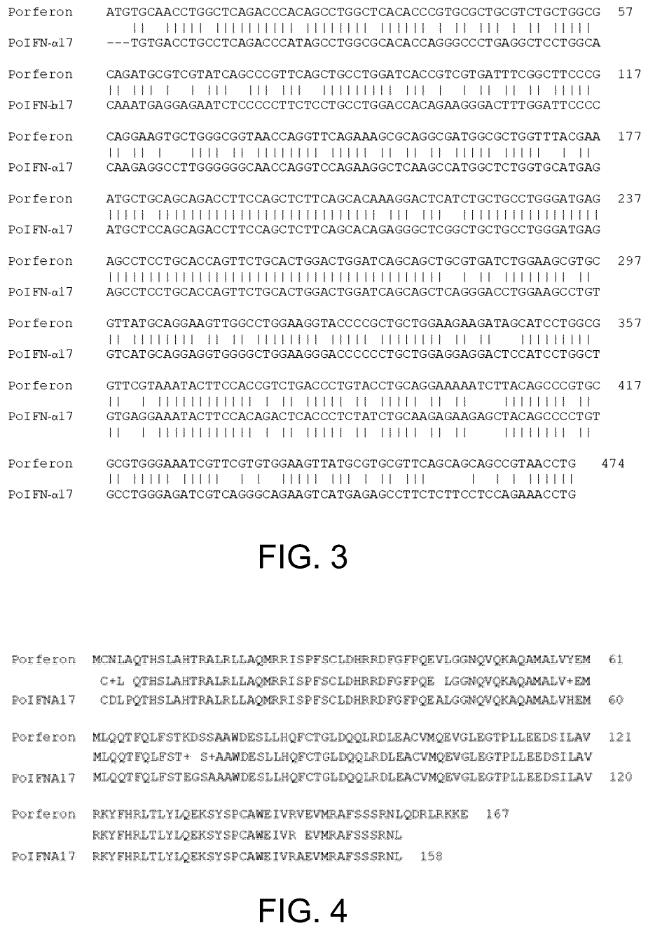

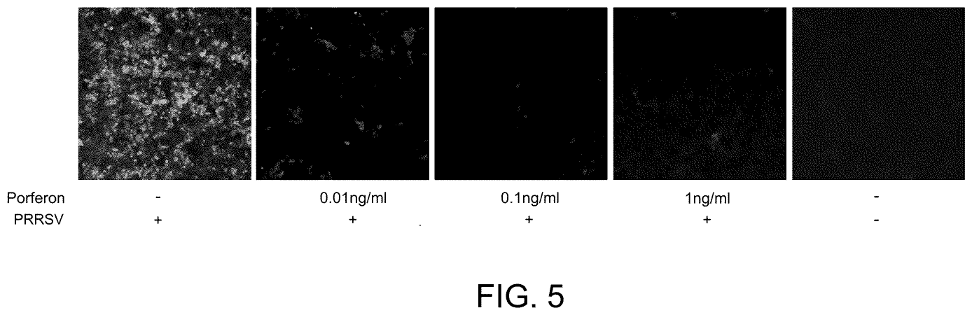

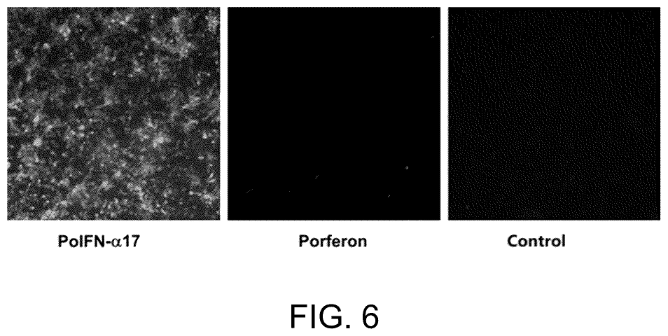

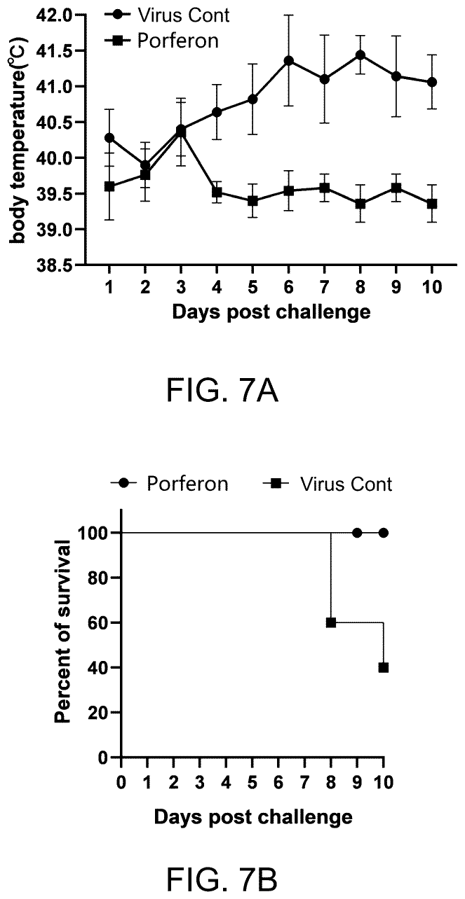

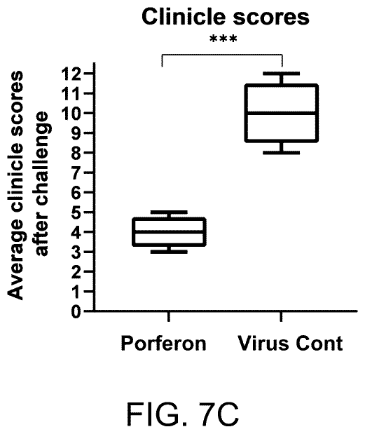

For 10 days post-infection, rectal temperatures were measured daily, and piglets were monitored for survival rates and clinical symptoms. The results are shown in FIGS. 7A, 7B and 7C. Statistical analysis revealed the following: 1). In the virus control group, infected piglets developed high fever typical of PRRSV infection (FIG. 7A) along with loss of appetite, lethargy, coarse hair coat, respiratory distress, periorbital edema, and mild diarrhea; 2). Over the 10-day observation period, 3 out of 5 piglets in the virus control group succumbed to infection (FIG. 7B); 3). In contrast, Porferon-treated piglets exhibited no fever, no significant clinical symptoms, and all remained healthy and survived; and 4). Comprehensive clinical scoring demonstrated that symptoms in the Porferon-treated group were significantly alleviated compared to the virus control group (FIG. 7C). These findings strongly indicate that Porferon effectively mitigates PRRSV-induced symptoms.

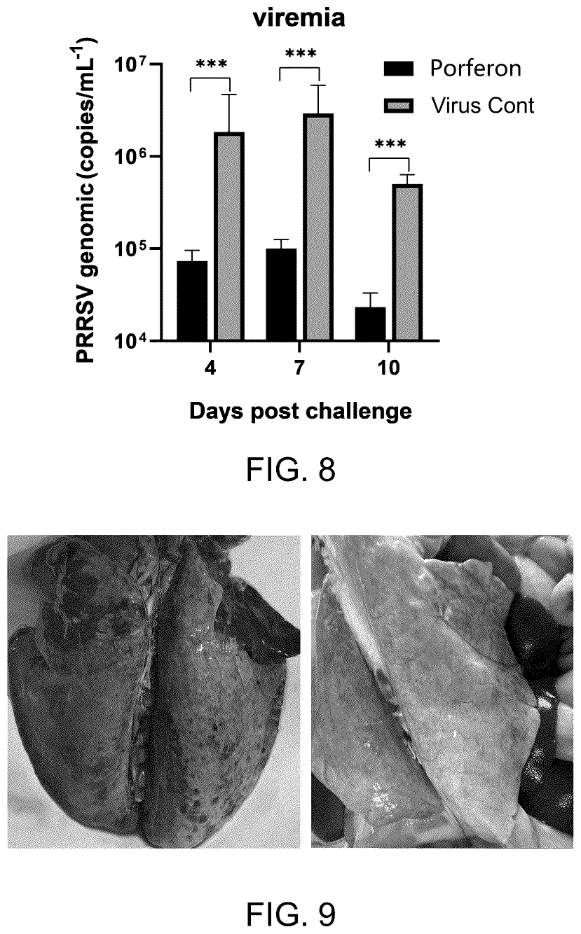

Blood samples were collected on Days 1, 4, 7, and 10 post-infection, and qRT-PCR was performed to determine PRRSV genomic cDNA copy numbers. The results are shown in FIG. 8. As illustrated in FIG. 8, Porferon-treated piglets exhibited significantly lower viral loads in serum between Days 4 and 10 compared to the virus control group, indicating that Porferon markedly reduces PRRSV replication in vivo.

On Day 10 post-infection, all animals were euthanized and necropsied to examine lung pathology in each group. The results are presented in FIG. 9. As shown in FIG. 9: 1). Virus control group piglets exhibited severe lung consolidation and hemorrhage, displaying the characteristic gross lesions of PRRSV infection (“blue ear disease”); and 2). Porferon-treated piglets only showed mild edema, with no hemorrhage or consolidation observed.

What is claimed is:

1. A porcine interferon-like recombinant protein, selected from one of the following:
A1) a protein having an amino acid sequence as shown in SEQ ID NO: 1;
A2) a fusion protein obtained by linking a tag to the N-terminal and/or C-terminal of the protein having the amino acid sequence as shown in SEQ ID NO: 1;
A3) a protein with one or more amino acid substitutions, deletions, and/or additions based on the amino acid sequence as shown in SEQ ID NO: 1, while retaining the same function;
A4) a protein having at least 85% sequence homology to the amino acid sequence as shown in SEQ ID NO: 1, while maintaining the same function;
A5) a mutated protein with 1 to 7 amino acid residue mutations compared to amino acid sequences of 17 porcine natural IFN-α subtypes, wherein sites of the mutations are selected from P(V)4A, A42V, H58Y, E71K, G72D, A74S, and A148V of the amino acid sequence as shown in SEQ ID NO: 1;
A6) a modified protein obtained by pharmaceutically acceptable protein modifications of the protein having the amino acid sequence as shown in SEQ ID NO: 1.

2. A biological material related to the porcine interferon-like recombinant protein of claim 1, selected from one of the following:
B1) a nucleic acid molecule encoding the porcine interferon-like recombinant protein;
B2) an expression cassette containing the nucleic acid molecule in B1;
B3) a recombinant vector containing the nucleic acid molecule in B1 or the expression cassette in B2;
B4) a recombinant microorganism containing the nucleic acid molecule in B1, the expression cassette in B2, or the recombinant vector in B3;
B5) an mRNA containing the nucleic acid molecule in B1;
B6) an adjuvant containing the nucleic acid molecule in B1;
B7) a vaccine containing the nucleic acid molecule in B1.

3. The biological material of claim 2, wherein a coding sequence of the nucleic acid molecule in B1 is as shown in SEQ ID NO: 2.

4. A pharmaceutical composition for preventing or treating a viral infection in pigs, comprising the porcine interferon-like recombinant protein of claim 1; or a biological material related to the porcine interferon-like recombinant protein,
wherein the biological material is selected from one of the following:
B1) a nucleic acid molecule encoding the porcine interferon-like recombinant protein;
B2) an expression cassette containing the nucleic acid molecule in B1;
B3) a recombinant vector containing the nucleic acid molecule in B1 or the expression cassette in B2;
B4) a recombinant microorganism containing the nucleic acid molecule in B1, the expression cassette in B2, or the recombinant vector in B3;
B5) an mRNA containing the nucleic acid molecule in B1;
B6) an adjuvant containing the nucleic acid molecule in B1;
B7) a vaccine containing the nucleic acid molecule in B1,
wherein virus causing the viral infection includes at least one of vesicular stomatitis virus, pseudorabies virus, classical swine fever virus, foot-and-mouth disease virus, porcine reproductive and respiratory syndrome virus (PRRSV), African swine fever virus, swine influenza virus, porcine circovirus, porcine adenovirus, porcine epidemic diarrhea virus, transmissible gastroenteritis virus, porcine delta coronavirus, porcine acute diarrhea syndrome coronavirus, swinepox virus, porcine parvovirus, Japanese encephalitis virus, porcine encephalomyocarditis virus, porcine enterovirus, porcine alphavirus encephalitis virus, porcine orthoreovirus, Seneca Valley virus, porcine measles virus, and porcine cytomegalovirus.

5. A pharmaceutical composition for preventing or treating a bacterial infection in pigs, comprising the porcine interferon-like recombinant protein of claim 1; or a biological material related to the porcine interferon-like recombinant protein,
wherein the biological material is selected from one of the following:
B1) a nucleic acid molecule encoding the porcine interferon-like recombinant protein;
B2) an expression cassette containing the nucleic acid molecule in B1;
B3) a recombinant vector containing the nucleic acid molecule in B1 or the expression cassette in B2;
B4) a recombinant microorganism containing the nucleic acid molecule in B1, the expression cassette in B2, or the recombinant vector in B3;
B5) an mRNA containing the nucleic acid molecule in B1;
B6) an adjuvant containing the nucleic acid molecule in B1;
B7) a vaccine containing the nucleic acid molecule in B1,
wherein bacteria causing the bacterial infection include at least one of Streptococcus suis, Escherichia coli, and Salmonella spp.

6. A pharmaceutical composition for preventing or treating a parasitic disease in pigs, comprising the porcine interferon-like recombinant protein of claim 1; or a biological material related to the porcine interferon-like recombinant protein,
wherein the biological material is selected from one of the following:
B1) a nucleic acid molecule encoding the porcine interferon-like recombinant protein;
B2) an expression cassette containing the nucleic acid molecule in B1;
B3) a recombinant vector containing the nucleic acid molecule in B1 or the expression cassette in B2;
B4) a recombinant microorganism containing the nucleic acid molecule in B1, the expression cassette in B2, or the recombinant vector in B3;
B5) an mRNA containing the nucleic acid molecule in B1;
B6) an adjuvant containing the nucleic acid molecule in B1;
B7) a vaccine containing the nucleic acid molecule in B1,
wherein parasite causing the parasitic diseases includes at least one of Toxoplasma gondii, Sarcoptes scabiei, and Trichinella spiralis.

7. An immune adjuvant of a vaccine, comprising the porcine interferon-like recombinant protein of claim 1; or a biological material related to the porcine interferon-like recombinant protein,
wherein the vaccine is for preventing infections caused by porcine viruses, bacteria, and/or parasites,
wherein the biological material is selected from one of the following:
B1) a nucleic acid molecule encoding the porcine interferon-like recombinant protein;
B2) an expression cassette containing the nucleic acid molecule in B1;
B3) a recombinant vector containing the nucleic acid molecule in B1 or the expression cassette in B2;
B4) a recombinant microorganism containing the nucleic acid molecule in B1, the expression cassette in B2, or the recombinant vector in B3;
B5) an mRNA containing the nucleic acid molecule in B1;
B6) an adjuvant containing the nucleic acid molecule in B1;
B7) a vaccine containing the nucleic acid molecule in B1,
wherein the porcine viruses include at least one of vesicular stomatitis virus, pseudorabies virus, classical swine fever virus, foot-and-mouth disease virus, porcine reproductive and respiratory syndrome virus (PRRSV), African swine fever virus, swine influenza virus, porcine circovirus, porcine adenovirus, porcine epidemic diarrhea virus, transmissible gastroenteritis virus, porcine delta coronavirus, porcine acute diarrhea syndrome coronavirus, swinepox virus, porcine parvovirus, Japanese encephalitis virus, porcine encephalomyocarditis virus, porcine enterovirus, porcine alphavirus encephalitis virus, porcine orthoreovirus, Seneca Valley virus, porcine measles virus, and porcine cytomegalovirus,
wherein the bacteria include at least one of Streptococcus suis, Escherichia coli, and Salmonella spp., and
wherein the parasites include at least one of Toxoplasma gondii, Sarcoptes scabiei, and Trichinella spiralis.

8. An animal feed, comprising the porcine interferon-like recombinant protein of claim 1; or a biological material related to the porcine interferon-like recombinant protein,
wherein the biological material is selected from one of the following:
B1) a nucleic acid molecule encoding the porcine interferon-like recombinant protein;
B2) an expression cassette containing the nucleic acid molecule in B1;
B3) a recombinant vector containing the nucleic acid molecule in B1 or the expression cassette in B2;
B4) a recombinant microorganism containing the nucleic acid molecule in B1, the expression cassette in B2, or the recombinant vector in B3;
B5) an mRNA containing the nucleic acid molecule in B1;
B6) an adjuvant containing the nucleic acid molecule in B1;
B7) a vaccine containing the nucleic acid molecule in B1.

9. A probiotic formulation, comprising the porcine interferon-like recombinant protein of claim 1; or a biological material related to the porcine interferon-like recombinant protein,
wherein the biological material is selected from one of the following:
B1) a nucleic acid molecule encoding the porcine interferon-like recombinant protein;
B2) an expression cassette containing the nucleic acid molecule in B1;
B3) a recombinant vector containing the nucleic acid molecule in B1 or the expression cassette in B2;
B4) a recombinant microorganism containing the nucleic acid molecule in B1, the expression cassette in B2, or the recombinant vector in B3;
B5) an mRNA containing the nucleic acid molecule in B1;
B6) an adjuvant containing the nucleic acid molecule in B1;
B7) a vaccine containing the nucleic acid molecule in B1.

10. An mRNA-based product, comprising the porcine interferon-like recombinant protein of claim 1; or a biological material related to the porcine interferon-like recombinant protein,
wherein the biological material is selected from one of the following:
B1) a nucleic acid molecule encoding the porcine interferon-like recombinant protein;
B2) an expression cassette containing the nucleic acid molecule in B1;
B3) a recombinant vector containing the nucleic acid molecule in B1 or the expression cassette in B2;
B4) a recombinant microorganism containing the nucleic acid molecule in B1, the expression cassette in B2, or the recombinant vector in B3;
B5) an mRNA containing the nucleic acid molecule in B1;
B6) an adjuvant containing the nucleic acid molecule in B1;
B7) a vaccine containing the nucleic acid molecule in B1,
wherein the mRNA-based product includes at least one of pharmaceutical compositions, vaccine adjuvants, feed additives, microbial formulations, diagnostic reagents, and reagent kits.

11. A product for modulating or enhancing porcine immunity, comprising the porcine interferon-like recombinant protein of claim 1; or a biological material related to the porcine interferon-like recombinant protein,
wherein the biological material is selected from one of the following:
B1) a nucleic acid molecule encoding the porcine interferon-like recombinant protein;
B2) an expression cassette containing the nucleic acid molecule in B1;
B3) a recombinant vector containing the nucleic acid molecule in B1 or the expression cassette in B2;
B4) a recombinant microorganism containing the nucleic acid molecule in B1, the expression cassette in B2, or the recombinant vector in B3;
B5) an mRNA containing the nucleic acid molecule in B1;
B6) an adjuvant containing the nucleic acid molecule in B1;
B7) a vaccine containing the nucleic acid molecule in B1,
wherein the product for modulating or enhancing porcine immunity includes at least one of pharmaceutical compositions, vaccine adjuvants, feed additives, microbial formulations, diagnostic reagents, and reagent kits.

12. The pharmaceutical composition of claim 4, further comprising a pharmaceutically acceptable carrier,
wherein the pharmaceutically acceptable carrier includes at least one of diluents, excipients, fillers, binders, wetting agents, disintegrants, absorption enhancers, surfactants, humectants, lubricants, adsorption carriers, and encapsulation agents; and
wherein the pharmaceutical composition is administered by injection, spraying, nasal drops, eye drops, transdermal absorption, physical or chemical mediation, or in combination with other substances for controlled delivery.

13. The pharmaceutical composition of claim 5, further comprising a pharmaceutically acceptable carrier, wherein the pharmaceutically acceptable carrier includes at least one of diluents, excipients, fillers, binders, wetting agents, disintegrants, absorption enhancers, surfactants, humectants, lubricants, adsorption carriers, and encapsulation agents; and
wherein the pharmaceutical composition is administered by injection, spraying, nasal drops, eye drops, transdermal absorption, physical or chemical mediation, or in combination with other substances for controlled delivery.

14. The pharmaceutical composition of claim 6, further comprising a pharmaceutically acceptable carrier, wherein the pharmaceutically acceptable carrier includes at least one of diluents, excipients, fillers, binders, wetting agents, disintegrants, absorption enhancers, surfactants, humectants, lubricants, adsorption carriers, and encapsulation agents; and
wherein the pharmaceutical composition is administered by injection, spraying, nasal drops, eye drops, transdermal absorption, physical or chemical mediation, or in combination with other substances for controlled delivery.

15. A feed additive, comprising the porcine interferon-like recombinant protein of claim 1; or a biological material related to the porcine interferon-like recombinant protein,
wherein the biological material is selected from one of the following:
B1) a nucleic acid molecule encoding the porcine interferon-like recombinant protein;
B2) an expression cassette containing the nucleic acid molecule in B1;
B3) a recombinant vector containing the nucleic acid molecule in B1 or the expression cassette in B2;
B4) a recombinant microorganism containing the nucleic acid molecule in B1, the expression cassette in B2, or the recombinant vector in B3;
B5) an mRNA containing the nucleic acid molecule in B1;
B6) an adjuvant containing the nucleic acid molecule in B1;
B7) a vaccine containing the nucleic acid molecule in B1.

16. A method for preparing the porcine interferon-like recombinant protein of claim 1, comprising:
constructing a recombinant expression vector containing a nucleic acid molecule encoding the porcine interferon-like recombinant protein;
introducing the recombinant expression vector into a host cell to obtain a recombinant host cell;
culturing the recombinant host cell to express the porcine interferon-like recombinant protein;
isolating and purifying the porcine interferon-like recombinant protein.


SEQUENCE LISTING (27 sequences)

SEQ ID NO: 1 - protein, 159 aa, synthetic construct
MCNLAQTHSLAHTRALRLLAQMRRISPFSCLDHRRDFGFPQEVLGGNQVQKAQAMALVYEMLQQTFQLFSTKDSSAAWDESLLHQFCTGLDQQLRDLEACVMQEVGLEGTPLLEEDSILAVRKYFHRLTLYLQEKSYSPCAWEIVRVEVMRAFSSSRNL

SEQ ID NO: 2 - DNA, 477 bp, synthetic construct
  source 1..477: mol_type = other DNA
atgtgcaacctggctcagacccacagcctggctcacacccgtgcgctgcgtctgctggcgcagatgcgtcgtatcagcccgttcagctgcctggatcaccgtcgtgatttcggcttcccgcaggaagtgctgggcggtaaccaggttcagaaagcgcaggcgatggcgctggtttacgaaatgctgcagcagaccttccagctcttcagcacaaaggactcatctgctgcctgggatgagagcctcctgcaccagttctgcactggactggatcagcagctgcgtgatctggaagcgtgcgttatgcaggaagttggcctggaaggtaccccgctgctggaagaagatagcatcctggcggttcgtaaatacttccaccgtctgaccctgtacctgcaggaaaaatcttacagcccgtgcgcgtgggaaatcgttcgtgtggaagttatgcgtgcgttcagcagcagccgtaacctg

SEQ ID NO: 3 - protein, 5 aa, synthetic construct
RRRRR

SEQ ID NO: 4 - protein, 6 aa, synthetic construct
HHHHHH

SEQ ID NO: 5 - protein, 8 aa, synthetic construct
HHHHHHHH

SEQ ID NO: 6 - protein, 8 aa, synthetic construct
DYKDDDDK

SEQ ID NO: 7 - protein, 8 aa, synthetic construct
WSHPQFEK

SEQ ID NO: 8 - protein, 10 aa, synthetic construct
EQKLISEEDL

SEQ ID NO: 9 - DNA, 75 bp, synthetic construct
  source 1..75: mol_type = other DNA
cgaagcattggttaaaaattaaggaggaaggatccatggccccawcctcarcyytcytcayrgycctggtgctac

SEQ ID NO: 10 - DNA, 60 bp, synthetic construct
  source 1..60: mol_type = other DNA
cytcayrgycctggtgctactcagctrcaakgccatctrctstctgggctgcraccygsy

SEQ ID NO: 11 - DNA, 59 bp, synthetic construct
  source 1..59: mol_type = other DNA
tstctgggctgcraccygsytcagaccyacagcctggctcacaccagggccctgaggcy

SEQ ID NO: 12 - DNA, 60 bp, synthetic construct
  source 1..60: mol_type = other DNA
acaccagggccctgaggcycctggcacaaatgaggagaatctcccccttctcctgcctgg

SEQ ID NO: 13 - DNA, 60 bp, synthetic construct
  source 1..60: mol_type = other DNA
ctcccccttctcctgcctggaccacagaarggactttggatycccycawgaggcyttkgg

SEQ ID NO: 14 - DNA, 60 bp, synthetic construct
  source 1..60: mol_type = other DNA
tycccycawgaggcyttkgggggcaaccaggtccagaaggctcaagccatggctctggtg

SEQ ID NO: 15 - DNA, 60 bp, synthetic construct
  source 1..60: mol_type = other DNA
ctcaagccatggctctggtgcatgagatgctccagcagacctyccagctcttcagcacag

SEQ ID NO: 16 - DNA, 60 bp, synthetic construct
  source 1..60: mol_type = other DNA
ctyccagctcttcagcacagagggctcrgctgctgcctggratgasagcctcctgcacca

SEQ ID NO: 17 - DNA, 60 bp, synthetic construct
  source 1..60: mol_type = other DNA
ratgasagcctcctgcaccagttctgcactggactggatcagcagctcagggacctggaa

SEQ ID NO: 18 - DNA, 60 bp, synthetic construct
  source 1..60: mol_type = other DNA
agcagctcagggacctggaagcctgtgtcatrcaggaggysgggctggaagggacscccc

SEQ ID NO: 19 - DNA, 60 bp, synthetic construct
  source 1..60: mol_type = other DNA
sgggctggaagggacscccctgctggaggaggrctccatcctggctgtgaggaaatactt

SEQ ID NO: 20 - DNA, 60 bp, synthetic construct
  source 1..60: mol_type = other DNA
ctggctgtgaggaaatacttccacagactcaycctctatctgcargagaagarctacagc

SEQ ID NO: 21 - DNA, 61 bp, synthetic construct
  source 1..61: mol_type = other DNA
tgcargagaagarctacagccctctgtgcctgggagatcrtcagggcagaagtcatgasak

SEQ ID NO: 22 - DNA, 61 bp, synthetic construct
  source 1..61: mol_type = other DNA
cagggcagaagtcatgasakctcttctcttcctccasamacctgcaagacagactcaggar

SEQ ID NO: 23 - DNA, 82 bp, synthetic construct
  source 1..82: mol_type = other DNA
ctgcaagacagactcaggargaaggagctcgagcaccaccatcaccatcaccatcactaagtgaattctgcagatatccatc

SEQ ID NO: 24 - DNA, 18 bp, synthetic construct
  source 1..18: mol_type = other DNA
accatgaaggtgacgctc

SEQ ID NO: 25 - DNA, 17 bp, synthetic construct
  source 1..17: mol_type = other DNA
aatcttctctcatccgc

SEQ ID NO: 26 - DNA, 22 bp, synthetic construct
  source 1..22: mol_type = other DNA
gaaggctttggggtgtgtgata

SEQ ID NO: 27 - DNA, 18 bp, synthetic construct
  source 1..18: mol_type = other DNA
tggcggatgagagaagat